(19) United States
(12) Patent Application Publication
(10) Pub. No.: US 2007/0099194 A1
(43) Pub. Date: May 3, 2007

(54) Quality control of probe attachment on electrodes

(76) Inventors: Xing Yang, San Diego, CA (US); Qun Zhu, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/264,996

(22) Filed: Nov. 2, 2005

(51) Int. Cl. C12Q 1/68; C12M 1/34

(52) U.S. Cl. 435/6; 435/287.2

(57) ABSTRACT

The present disclosure relates to methods of quantifying nucleic acid molecules immobilized on an electrode of an electrochemical assay chip. Such quantification can be used in testing and quality control of assay chips. Materials and instrumentation useful in performing such techniques are also disclosed.

### BACKGROUND OF THE INVENTION

1. Field of the Invention

The present invention relates to methods and apparatus for nucleic acid detection. In particular, techniques for quantifying nucleic acids on an electrode surface are useful for testing and quality control.

2. Description of the Related Art

Numerous references disclose the detection of nucleic acid hybridization by quantifying a current carried by a redox moiety at an electrode. Such techniques are described in: A. B. Steel et al., Electrochemical Quantitation of DNA Immobilized on Gold, Anal. Chem. 70:4670-77 (1998); U.S. patent application Ser. No. 10/429,291 filed May 2, 2003 (published as US-2004-0086894-A1); U.S. patent application Ser. No. 10/429,293 filed May 2, 2003 (published as US-2004-0086895-A1); U.S. patent application Ser. No. 10/985,256 filed Nov. 10, 2004 (published as US-2005-0186590-A1); all of which are hereby expressly incorporated by reference. Ruthenium hexamine is one redox moiety that can be used; U.S. patent application Ser. No. 10/429,291 describes numerous other moieties with various redox potentials that can also be used.

When detecting a signal indicative of a target nucleic acid, it has been observed that the signal is proportional to the number of target nucleic acid molecules at the electrode surface. In the linear range, the number of targets is roughly proportional to the number of capture probes on the electrode surface. Accordingly, in conducting quality control on assay plates, it is advantageous to know the quantity of capture probes immobilized on the electrode surface.

There exists an unmet need in the art for a quick, reliable, and inexpensive quality control method to quantify immobilized capture probes and correlate target signals to probe signals.

### SUMMARY OF THE INVENTION

One aspect of the invention is a method of quantifying nucleic acid probes including: providing an electrode surface; immobilizing a plurality of nucleic acid probes on the electrode surface; providing a redox moiety capable of electrostatic association with the plurality of nucleic acid probes; and detecting a total electrical current transferred by the redox moiety, wherein the total electrical current comprises a diffusion current and an adsorption current, wherein the detection occurs at a frequency at which the adsorption current is greater than the diffusion current.

Another aspect of the invention is a method of evaluating the quality of a nucleic acid assay device including: providing a nucleic acid assay device comprising an electrode surface wherein a plurality of nucleic acid probes are immobilized on the electrode surface; quantitating the nucleic acid probes by detecting an electrical signal; hybridizing a plurality of nucleic acid targets to the nucleic acid probes; quantitating the nucleic acid targets by detecting an electrical signal; correlating the quantity of the nucleic acid probes with the quantity of the nucleic acid targets to evaluate the quality of the nucleic acid assay device.

Another aspect of the invention is a method for performing an electrochemical assay, including: providing an assay device containing at least one electrode having polynucleotide capture probe attached; providing a redox moiety in solution in contact with the electrode, wherein the redox moiety is capable of association with both the capture probe and with a polynucleotide target capable of hybridizing with the capture probe; quantitating the capture probe on the electrode by measuring current associated with redox interaction with the electrode using cyclic voltammetry at a frequency greater than 75 Hz; thereafter contacting the capture probe with a liquid to be analyzed for the presence of the polynucleotide target; measuring a target current associated with interaction between the electrode and redox moiety associated with the polynucleotide target; and calibrating the result of the target current measurement with reference to the quantity of polynucleotide capture probe on the electrode.

Another aspect of the present invention is a method for performing an electrochemical assay, including the steps of: providing an assay device having a capture probe attached to an electrode; providing device-specific data reflective of the quantity of capture probe on the electrode, as measured electrochemically; performing an electrochemical assay to detect an analyte, thereby generating a value reflective of the quantity of the analyte; and providing a result of the assay reflecting interpretation of the result in light of the device-specific data.

### BRIEF DESCRIPTION OF THE DRAWINGS

FIG. 1 shows a cross section of an electrode subject to adsorption current and diffusion current.

FIG. 2 shows scatter plots of target signals plotted against probe signals at three different frequencies.

FIG. 3 shows a theoretical perfect linear relationship between probe signal and hybrid signal.

FIG. 4 is a quality control chart that reports the results of testing numerous electrodes.

### DETAILED DESCRIPTION OF THE PREFERRED EMBODIMENT

In conducting quality control testing on nucleic acid hybridization based products, it is advantageous to be able to quantify the number of capture probes immobilized on an electrode surface in order to verify usability of the product for detection of nucleic acid hybridization events. The number of capture probes immobilized on the surface has a direct correlation to the signal generated in an electrochemical reaction used to determine the occurrence of target nucleic acid hybridization to the capture probes.

Common techniques for quantifying capture probes include the use of fluorescent and radioactive labels. However, these methods have certain disadvantages. For example, fluorescent labeling works poorly with some substrate materials, such as certain plastics and fiberglass, because they generate autofluorescence. Also, some electrode materials, such as carbon, strongly absorb fluorescence signals. Further, labeling techniques generally involve modification of some or all of the capture probes; such modification can interfere with subsequent hybridization and/or electrochemical detection. It is highly desirable to have a reliable quantification assay that does not require changing the immobilized capture probes on the substrate.

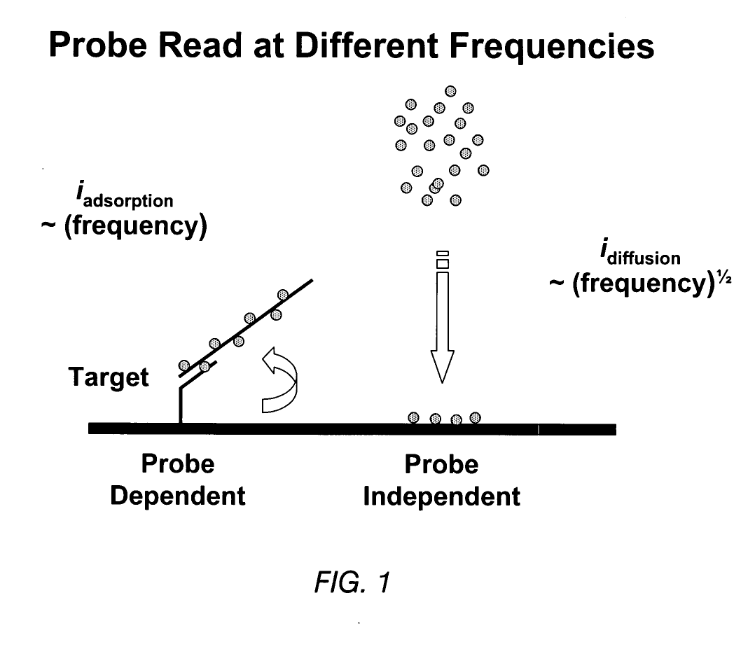

The detectable current signal in an electrochemical assay arises from two main sources: adsorption current and diffusion current. These phenomena are illustrated in FIG. 1. The adsorption current (i_{adorption}, shown on the left) is due to the presence of redox moieties (such as ruthenium hexaamine) that associate electrostatically with the phosphate backbone of nucleic acids, and undergo a redox reaction at the electrode surface. In contrast, the diffusion current (i_{diffusion}, shown on the right) is due to redox moieties traveling through the assay medium at random, contacting the electrode surface irrespective of the presence or absence of any nucleic acid, and undergoing a redox reaction at the electrode surface to generate a current. This latter type of current can be considered background noise, because it occurs regardless of the presence or absence of capture probes and/or target nucleic acids. In determining whether hybridization of the target nucleic acid to the capture probes has occurred, one looks to the adsorption current. Since the adsorption current is dependent on the quantity of nucleic acid present, target nucleic acid hybridized to a capture probe will generate a larger signal than the capture probe alone, thus indicating that hybridization has occurred.

In polynucleotide assays in which electrochemical signal is a function of the total nucleic acid concentration on or near the electrode surface (of whatever origin, e.g., capture probe and/or target nucleic acid), the adsorption current is proportional to the number of nucleic acid strands on or near the electrode surface, whereas the diffusion current is independent of the presence of nucleic acid strands on or near the surface. When using square wave voltammetry readout parameters at low frequency (for example, less than about 50 Hz), the diffusion current signal is dominant. At these low frequencies, the adsorption current, which forms the basis of the detection assay, is overshadowed so that no correlation between target signals and probe signals can be observed.

As reported in Faulkner, et al., Electrochemical Methods: Fundamentals and Applications, 2nd ed., 2001, herein expressly incorporated by reference in its entirety, the diffusion current is approximately proportional to the square root of the readout frequency, while the adsorption current is linearly proportional to the readout frequency. Accordingly, when the readout frequency is increased, the adsorption current increases much more quickly than diffusion current. For example, assume that at a 15 Hz readout frequency, the observed diffusion current is about 6 nA, and the observed adsorption current is about 2 nA. If the readout frequency is increased to 300 Hz, the diffusion current is approximately 27 nA and the adsorption current is approximately 40 nA. Because the percentage of total current that is due to adsorption current increases with frequency, the adsorption current can be made the dominant part of the signal by increasing the readout frequency.

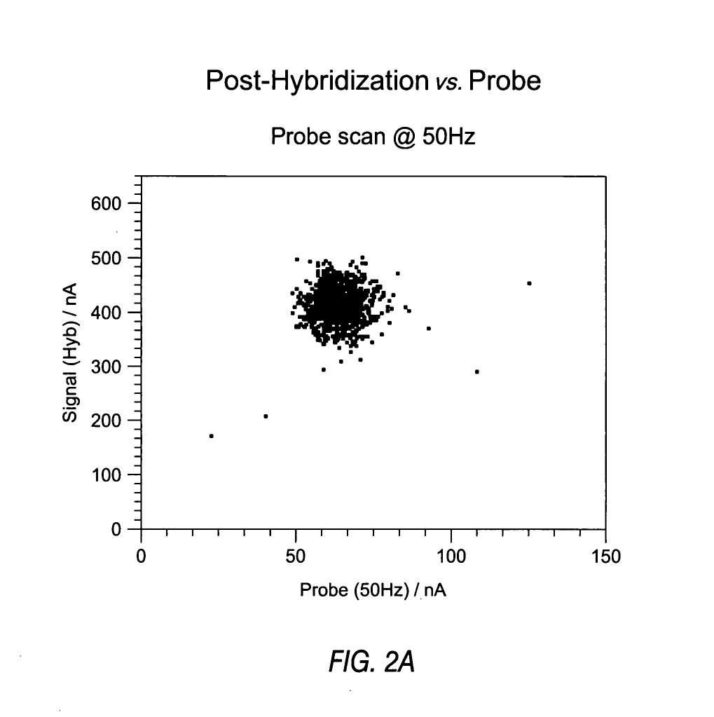

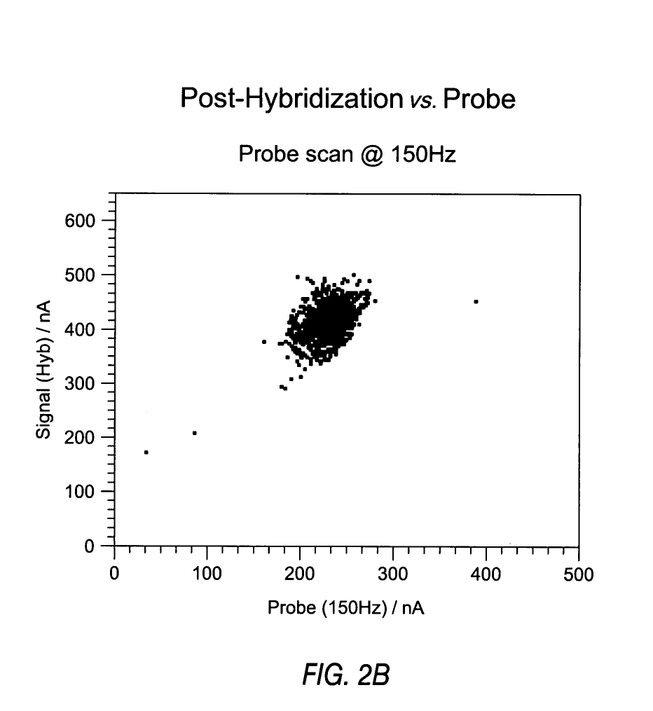

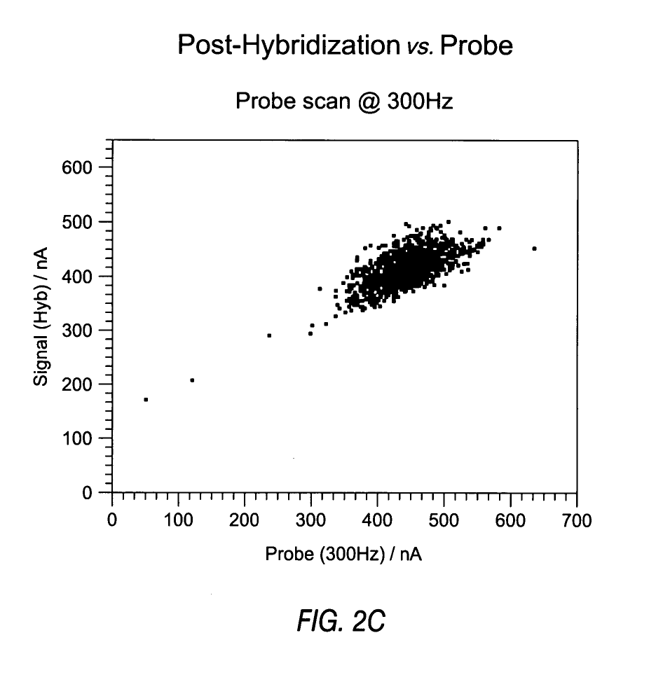

FIG. 2 shows data obtained from detecting current at three different frequencies, 50 Hz, 150 Hz, and 300 Hz. In the plots, current from the probes appears on the x-axis and current from both probes and targets (the targets are hybridized to immobilized probes) appears on the y-axis. For each axis, the scale is in nanoamps (nA). At each frequency, current is detected before hybridization to obtain the x-axis data, and following hybridization of targets to the probes to obtain the y-axis data. Ruthenium hexaamine associates electrostatically with both target nucleic acids and capture probes, which means that both produce a detectable current at the electrode surface.

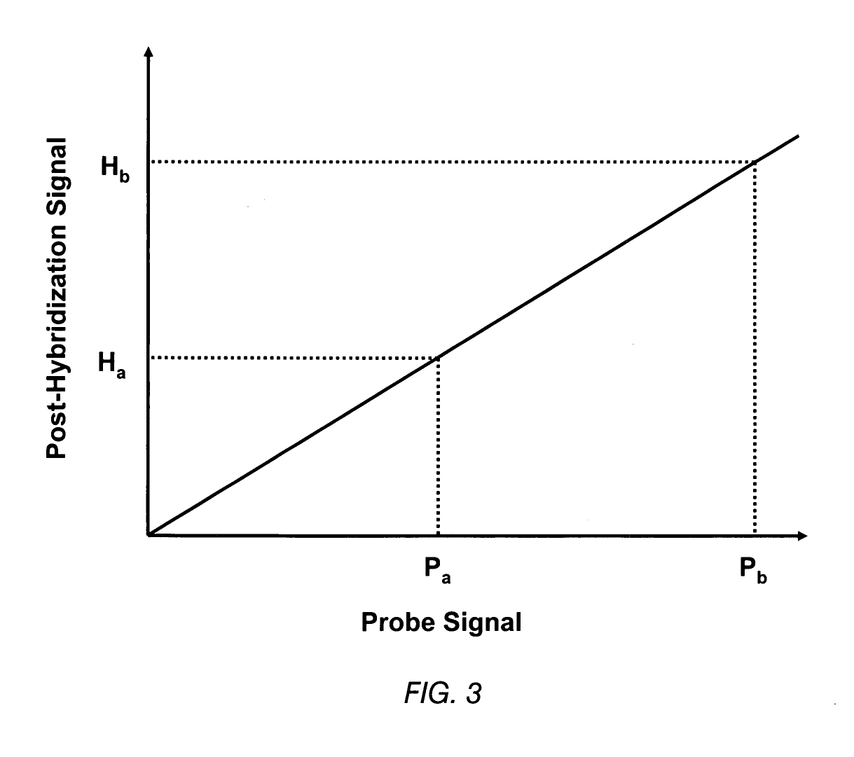

As shown in FIG. 2, the relationship between the capture probe and target nucleic acid signals is not very clear at 50 Hz. In the absence of diffusion current, one would expect that a linear relationship would exist between the current from the capture probes and the current from the hybridization complex of capture probes and target nucleic acids. At 50 Hz, however, the diffusion current overshadows the adsorption current and obscures the linear relationship. However, as the frequency is increased to 150 Hz, and then 300 Hz, the linear relationship becomes more discernible, because a greater percentage of the total current is due to adsorption current. FIG. 3 shows a theoretical, perfect linear relationship between the probe signal and the hybrid signal.

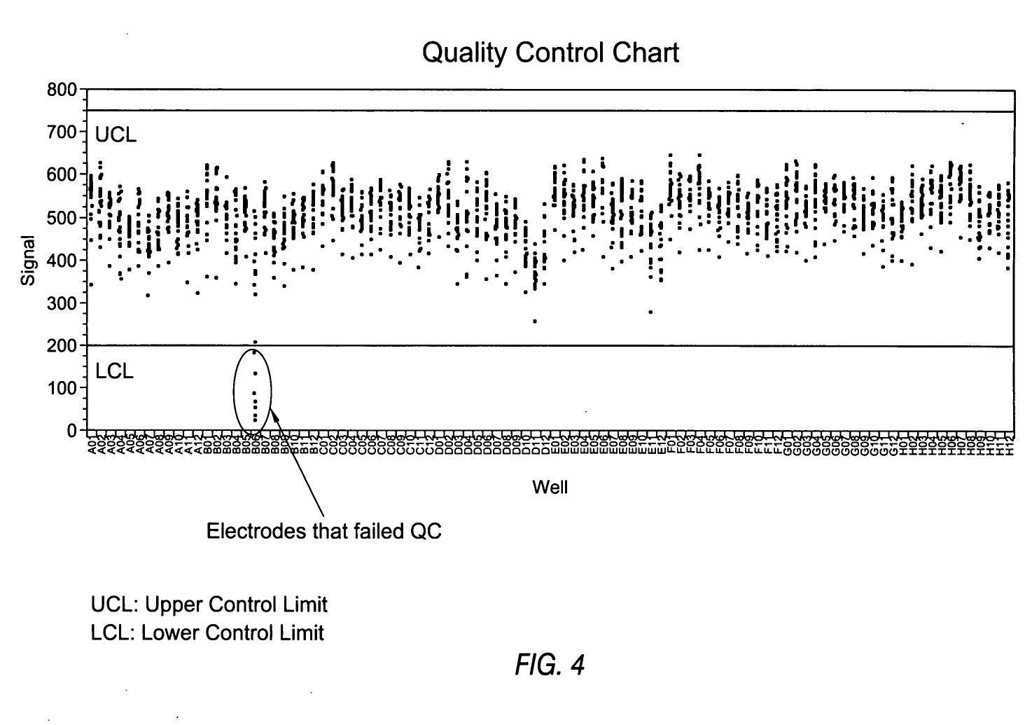

The linear relationship permits a user to translate an acceptable range for the post-hybridization signal into a quality control range for the probe signal. FIG. 4 shows quality control (QC) criteria implemented. In FIG. 4, electrodes are deemed to have passed QC if the signal is above the lower control limit (LCL) of 200 nA and below the upper control limit (UCL) of 750 nA. Several electrodes in well B06 failed QC because the observed signal was lower than the LCL. Those of skill in the art will appreciate that other LCLs and UCLs can be established based on a cost-benefit analysis in which the cost of rejecting some inventory is compared with the benefit of having electrodes that meet a certain quality level.

Those of skill in the art will also appreciate that relatively high readout frequencies are advantageous in developing a quality control testing regimen. For example, frequencies above 50 Hz are preferred. In particular, useful frequencies include 100 Hz, 150 Hz, 200 Hz, 250 Hz, 300 Hz, 350 Hz, 400 Hz, 450 Hz, 500 Hz, and higher. However, it has been observed that the use of higher frequency results in higher currents. If current is too high, it can damage electrical circuitry. A preferred range for the probe signal current is approximately 200-750 nA. Thus, useful current levels include 200, 250, 300, 350, 400, 450, 500, 550, 600, 650, 700, and 750 nA.

Additionally, it is advantageous to set the quality control testing parameters so that the adsorption current is greater than the diffusion current. In some embodiments, the adsorption current is twice, three times, four times, five times, six times, seven times, eight times, nine times, or ten times as large as the diffusion current.

The invention can be used in several ways to optimize manufacturing and performance of an electrochemical assay. First, it can be used as part of a quality assurance program, to verify that a desired quantity of capture probe is attached to an electrode prior to packaging and shipping of the assay. In this manner, the assay devices sold by that manufacturer will meet accepted standards of uniformity and operability. Second, the data generated by the present invention could be used to pre-calibrate assays at the factory or manufacturing level. Having already quantified the signal-producing capture probe, indicia reflective of this information can be provided with the assay device or incorporated on or in the assay device, e.g., in the form of a label, barcode, package insert, or electronically-readable device, e.g., magnetic, printed code, embedded code, RF tag, etc.

Alternatively, the invention can be used to provide a contemporaneous quality control or calibration at the time of the assay. Once the assay device is connected to an assay reader, electronic controls in the reader (not shown) can measure the quantity of capture probe attached to each electrode. This information can be used, in turn, to calibrate or interpret the assay results, e.g., leading to better quantification of those results.

### EXAMPLE 1

### Quality Control Protocol

The following materials and methods were used to produce the readouts shown in FIG. 2.

Chips having 96 wells with 20 carbon electrodes per well were used. Nucleic acid probes were immobilized on the electrodes and complementary target strands were added for the hybridization portion of the assay. The washing and detection buffer used was 10 mM HEPES/10 mM NaCl/5 μM ruthenium hexaamine. The hybridization buffer was 11 mM HEPES/1.1 M LiCl. An ePlex™ reader from GeneOhm Sciences, Inc. was used and the scan frequencies were 50 Hz, 150 Hz, and 300 Hz in a window of −100 mV to −600 mV (vs. Ag/AgCl), at an amplitude of −40 mV, and at steps of −10 mV.

What is claimed is:

1. A method of quantifying nucleic acid probes comprising:
providing an electrode surface;
immobilizing a plurality of nucleic acid probes on said electrode surface;
providing a redox moiety capable of electrostatic association with said plurality of nucleic acid probes; and
detecting a total electrical current transferred by said redox moiety, wherein said total electrical current comprises a diffusion current and an adsorption current, wherein said detection occurs at a frequency at which said adsorption current is greater than said diffusion current.

2. The method of claim 1 wherein said electrode surface comprises gold.

3. The method of claim 1 wherein said electrode surface comprises carbon.

4. The method of claim 1 wherein said redox moiety comprises ruthenium.

5. The method of claim 4 wherein said redox moiety is ruthenium hexamine.

6. The method of claim 1 wherein said frequency is greater than about 50 Hz.

7. The method of claim 1 wherein said frequency is greater than about 200 Hz.

8. The method of claim 1 wherein said frequency is about 300 Hz.

9. The method of claim 1 wherein said frequency is greater than about 300 Hz.

10. The method of claim 1 wherein said adsorption current is at least twice as large as said diffusion current.

11. A method of evaluating the quality of a nucleic acid assay device comprising:
providing a nucleic acid assay device comprising an electrode surface wherein a plurality of nucleic acid probes are immobilized on said electrode surface;
quantitating said nucleic acid probes by detecting an electrical signal;
hybridizing a plurality of nucleic acid targets to said nucleic acid probes;
quantitating said nucleic acid targets by detecting an electrical signal;
correlating the quantity of said nucleic acid probes with the quantity of said nucleic acid targets to evaluate the quality of the nucleic acid assay device.

12. A method for performing an electrochemical assay, comprising:
providing an assay device comprising at least one electrode having polynucleotide capture probe attached;
providing a redox moiety in solution in contact with the electrode, wherein the redox moiety is capable of association with both the capture probe and with a polynucleotide target capable of hybridizing with the capture probe;
quantitating the capture probe on the electrode by measuring current associated with redox interaction with the electrode using cyclic voltammetry at a frequency greater than 75 Hz;
thereafter contacting the capture probe with a liquid to be analyzed for the presence of the polynucleotide target;
measuring a target current associated with interaction between the electrode and redox moiety associated with the polynucleotide target; and
calibrating the result of the target current measurement with reference to the quantity of polynucleotide capture probe on the electrode.

13. A method for performing an electrochemical assay, comprising the steps of:
providing an assay device having a capture probe attached to an electrode;
providing device-specific data reflective of the quantity of capture probe on the electrode, as measured electrochemically;
performing an electrochemical assay to detect an analyte, thereby generating a value reflective of the quantity of the analyte; and
providing a result of the assay reflecting interpretation of the result in light of the device-specific data.
